(19) United States
(12) Patent Application Publication
(10) Pub. No.: US 2026/0137448 A1
(43) Pub. Date: May 21, 2026

(54) APPARATUS FOR LASER TREATMENT

(71) Applicant: Tecure, Inc., Busan (KR)

(72) Inventors: Gia Van TRUONG, Busan (KR); Hyejin KIM, Busan (KR)

(21) Appl. No.: 19/372,940

(22) Filed: Oct. 29, 2025

(30) Foreign Application Priority Data
Nov. 21, 2024 (KR) 10-2024-0167444
Jul. 23, 2025 (KR) 10-2025-0100019

(51) Int. Cl. A61B 18/24; A61B 17/00; A61B 18/00; A61B 18/22; A61B 34/20; A61M 25/01; A61M 25/06; A61M 25/10

(52) U.S. Cl. CPC A61B 18/24; A61B 34/20; A61B 2017/00035; A61B 2017/0011; A61B 2017/00907; A61B 2018/00023; A61B 2018/00184; A61B 2018/00285; A61B 2018/00488; A61B 2018/00791; A61B 2018/00875; A61B 2018/2255; A61B 2034/2061; A61M 25/0136; A61M 2025/0681; A61M 25/10181

(57) ABSTRACT

An apparatus for laser treatment, which may provide precise laser treatment in a body, the apparatus for laser treatment including: a light irradiation unit including an optical fiber; a catheter having an expansion fixing portion into which the optical fiber is inserted; a sensor monitoring unit including a plurality of sensors provided in the catheter; and a controller receiving measurement information from the sensor monitoring unit and controlling the light source Furthermore, the plurality of sensors include a shape information sensor inserted together with the optical fiber and configured to obtain internal shape information of the tubular tissue in a position of an optical fiber end to which light is irradiated, and in a cross-section, perpendicular to a longitudinal direction of the catheter, the shape information sensor is disposed in a central portion, together with the optical fiber.

### CROSS-REFERENCE TO RELATED APPLICATION(S)

This application claims benefit of priority to Korean Patent Application Nos. 10-2024-0167444 and 10-2025-0100019 filed on Nov. 21, 2024, and Jul. 23, 2025, in the Korean Intellectual Property Office, the disclosures of which are incorporated herein by reference in their entirety.

### BACKGROUND

### 1. Field

The present disclosure relates to an apparatus for laser treatment that irradiates a laser to tubular tissue.

### 2. Description of Related Art

Barrett's esophagus (BE) is a common digestive disorder, affecting approximately 1.6% to 6.8% of the general U.S. population, which occurs in approximately 10% of those experiencing acid reflux symptoms. Barrett's esophagus is a condition in which the flat, pink lining of the esophagus, which connects the mouth to the stomach, thickens and turns red due to damage from acid reflux. These changes are considered precancerous mucosal changes, and are associated with the development of esophageal adenocarcinoma, and symptoms are often associated with gastroesophageal reflux disease (GERD), which can include chest pain, heartburn, regurgitation, and dysphagia. GERD is a significant cause of Barrett's esophagus.

Endoscopic procedures have recently been introduced as an alternative to traditional surgery and long-term medication. Endoscopic treatment options for Barrett's esophagus (BE) focus on removing or ablating abnormal tissue in the esophagus to reduce the risk of developing esophageal adenocarcinoma. Several endoscopic treatments for Barrett's esophagus include radiofrequency ablation (RFA), cryotherapy, and endoscopic mucosal resection (EMR). The RFA uses radiofrequency current to generate heat and apply the heat to abnormal tissue in the esophagus.

RFA devices are often combined with balloons to deliver temperature-controlled thermal energy to the target tissue, thus ablating Barrett's esophagus tissue. However, clinically used endoscopic RFA procedures often carry short-term risks of bleeding, chest pain, and infection, as well as the risk of long-term complications such as recurrence, surgical correction, and strictures.

Cryotherapy is a non-contact procedure that directly injects liquid nitrogen at −196° C. through an endoscope onto the target lesion, freezing and destroying the tissue. Recently, cryoballoon ablation has emerged, and this procedure is a method of inflating a balloon in the BE segment and then locally spraying liquid nitrogen to perform ablating. However, this procedure remains unstable.

Furthermore, EMR is a minimally invasive procedure that removes abnormal tissue from the esophageal mucosa, and then excises lifted tissue using a special device attached to an endoscope. However, this procedure requires a high level of operator skill and a long operating time, and carries an increased risk of procedure-related complications such as chest pain, sore throat, severe bleeding, perforation, and stricture.

Endoscopic treatments may provide access to more patients and help reduce the global burden of disease. Current endoscopic treatments involve space-occupying devices and sutures or adhesions, and small intestine-specific endoscopic obesity and metabolic treatments have been developed.

Duodenal Mucosal Resurfacing (DMR) is a novel minimally invasive endoscopic procedure that removes the duodenal mucosa and induces regeneration to improve glucose metabolism and insulin sensitivity in patients with type 2 diabetes. In recent duodenal mucosal resurfacing procedures, the duodenal mucosa is removed using radiofrequency energy or hot water, resulting in re-epithelialization of the duodenal lining. While initial clinical trials have demonstrated DMR's safety and efficacy, further improvements are required.

Endoscopic devices are required for the treatment of BE and DMR, but existing endoscopic devices are not suitable for these procedures.

### SUMMARY

An aspect of the present disclosure is to provide an apparatus for laser treatment used in an endoscope device and capable of precisely irradiating light in various manners.

An aspect of the present disclosure is to provide an apparatus for laser treatment capable of providing precise phototherapy in a body.

The present disclosure provides the following apparatus for laser treatment to achieve the aspect described above.

In an embodiment, the present disclosure provides an apparatus for laser treatment including: a light irradiation unit including an optical fiber connected to a light source; a catheter having an expansion fixing portion into which the optical fiber is inserted, and which fixes a position thereof in a tubular tissue within a body; and a sensor monitoring unit including a plurality of sensors provided in the catheter; and a controller receiving measurement information from the sensor monitoring unit and controlling the light source of the light irradiation unit, and including a processor and a storage medium connected to the processor and storing a control program, and the plurality of sensors may include a shape information sensor inserted together with the optical fiber and configured to obtain internal shape information of the tubular tissue in a position of an optical fiber end to which light is irradiated.

In an embodiment, in a cross-section, perpendicular to a longitudinal direction of the catheter, the shape information sensor may be disposed in a central portion, together with the optical fiber, and the catheter may include a transparent tube surrounding the optical fiber and a delivery tube surrounding the transparent tube, the sensor monitoring unit may include a shape information sensor wire connected to the shape information sensor, and the shape information sensor wire may be disposed inside the delivery tube.

In an embodiment, the expansion fixing portion may include a balloon into which the optical fiber is inserted and which is inflated by a fluid, and the shape information sensor may include a piezoelectric sensor.

In an embodiment, the piezoelectric sensor may be disposed in plural in a longitudinal direction of the catheter within the balloon, and the piezoelectric sensor may be disposed further from an end of the catheter than the optical fiber end.

In an embodiment, the apparatus for laser treatment may further include a fluid management unit including a pump for supplying fluid to inflate the balloon and removing the supplied fluid, and the controller may control the light source and the amount of fluid supplied from the fluid management unit based on a measurement value of the piezoelectric sensor.

In an embodiment, the fluid management unit may include an air trap remover so as to remove an air trap inside the balloon and a cooler generating a cooling fluid for lowering a temperature of the fluid.

In an embodiment, the delivery tube may have a plurality of channels, a transparent tube may be disposed in one of the plurality of channels, and the shape information sensor wire may be disposed in another channel.

In an embodiment, the plurality of sensors may include a temperature sensor, and the temperature sensor may be attached to the balloon.

In an embodiment, the apparatus for laser treatment may further include a cooling liquid supply portion supplying a cooling liquid to the optical fiber end through a cooling channel disposed within the delivery tube, and the cooling channel may include an outlet disposed closer to an end of the catheter than the optical fiber end, the expansion fixing portion may include a wire basket configured to be contractible and expandable, and the optical fiber end may be disposed within the wire basket.

In an embodiment, the wire basket may include a plurality of wires, and at least a portion of the plurality of sensors may be attached to the plurality of wires.

In an embodiment, the sensor attached to the wire may include at least one of a pH measurement sensor or a mucosal impedance measurement sensor.

In an embodiment, the expansion fixing portion further may include a balloon inflated by fluid in a different position from the wire basket in a longitudinal direction of the catheter.

In an embodiment, the balloon may be disposed further from the end of the catheter than the wire basket.

In an embodiment, the light irradiation portion may include a glass cap covering the optical fiber end, and the optical fiber may be one of a diffusion optical fiber, a radial optical fiber and a side-firing fiber.

In an embodiment, the glass cap may have a circular or polygonal inner surface in a longitudinal cross-section of the optical fiber.

In an embodiment, the apparatus for laser treatment may further include: a handpiece manipulating a movement of the catheter, and the piezoelectric sensor may be cylindrical, the optical fiber may be a side-firing fiber, and the handpiece may include a rotational manipulation unit connected to the optical fiber and the piezoelectric sensor and rotating the optical fiber and the piezoelectric sensor.

In an embodiment, the present disclosure provides an apparatus for laser treatment including: a light irradiation unit including an optical fiber connected to a light source; a catheter including a flexible tip disposed in a tip portion thereof, a balloon disposed behind the flexible tip, into which the optical fiber is inserted, and which fixes a position in a tubular tissue within a body, and an endoscope unit disposed in back of the balloon; a piezoelectric sensor attached to the optical fiber within the balloon and configured to obtain diameter information of the tubular tissue behind an optical fiber end to which light is irradiated; and a fluid management unit including a pump for supplying fluid to inflate the balloon and removing the supplied fluid, and the piezoelectric sensor may be disposed in plural in a longitudinal direction of the catheter, the piezoelectric sensor may be disposed in a central portion, together with the optical fiber, in a cross-section, perpendicular to the longitudinal direction of the catheter, the catheter may include a transparent tube surrounding the optical fiber and a delivery tube surrounding the transparent tube, and the delivery tube may include a channel through which fluid passes, a channel in which the transparent tube is disposed, and a channel in which a wire connected to the piezoelectric sensor is disposed.

In an embodiment, the apparatus for laser treatment may include a controller connected to the light source connected to the light irradiation unit, the piezoelectric sensor and the fluid management unit, and the controller may control the light source and the fluid management unit based on a measurement value of the piezoelectric sensor.

In an embodiment, the apparatus for laser treatment may include a moving portion including a motor for moving the catheter, and the controller may be connected to the moving portion.

The present disclosure may provide an apparatus for laser treatment used in an endoscope device and capable of precisely irradiating light in various manners.

The present disclosure may provide an apparatus for laser treatment capable of providing precise phototherapy in a body.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 is a schematic diagram of an apparatus for laser treatment according to a first embodiment of the present disclosure;

FIG. 2 is a schematic diagram of a catheter in an apparatus for laser treatment according to the first embodiment of the present disclosure;

FIG. 3 is a schematic diagram of a catheter in an apparatus for laser treatment according to a modified embodiment of the first embodiment of the present disclosure;

FIG. 4 is a schematic diagram of an apparatus for laser treatment according to a second embodiment of the present disclosure;

FIG. 5 is a schematic diagram of a catheter in an apparatus for laser treatment according to a third embodiment of the present disclosure;

FIG. 6 is a schematic diagram of a catheter in an apparatus for laser treatment according to a fourth embodiment of the present disclosure;

FIG. 7 is a schematic diagram of an apparatus for laser treatment according to a fifth embodiment of the present disclosure;

FIG. 8 is a schematic diagram of a catheter in an apparatus for laser treatment according to a fifth embodiment of the present disclosure;

FIGS. 9A to 9C are schematic diagrams of a balloon in the first embodiment of the present disclosure;

FIGS. 10A to 10B are schematic diagrams of a wire basket in the present disclosure;

FIGS. 11A to 11C are schematic diagrams illustrating a modified embodiment in an arrangement of the wire basket and balloon in the present disclosure;

FIG. 12 is a schematic diagram of a catheter according to a sixth embodiment of the present disclosure;

FIG. 13A is a schematic diagram of a catheter according to a seventh embodiment of the present disclosure;

FIG. 13B is a schematic diagram of a modified embodiment of the seventh embodiment;

FIG. 14 is a schematic cross-sectional view of the catheter according to the seventh embodiment;

FIG. 15A is a schematic diagram of a catheter according to an eighth embodiment of the present disclosure;

FIG. 15B is a schematic diagram of a modified embodiment of the eighth embodiment;

FIGS. 16A and 16B are schematic cross-sectional views of the eighth embodiment of the present disclosure;

FIG. 17 is a schematic diagram of an optical fiber end portion of the present disclosure;

FIGS. 18A to 18D are schematic diagrams illustrating light irradiation according to a modified embodiment in a glass cap on an end of an optical fiber;

FIGS. 19A to 19D are simulation views of light irradiation according to changes in the glass cap on the optical fiber end;

FIGS. 20A to 20C are schematic diagrams illustrating tissues treated according to changes in the glass cap on the optical fiber end;

FIGS. 21A and 21B are photographs of tissues treated according to changes in the glass cap on the optical fiber end;

FIG. 22 is a perspective view of a first embodiment of a handpiece of an apparatus for laser treatment of the present disclosure;

FIG. 23 is a schematic diagram of a first embodiment of a handpiece of an apparatus for laser treatment of the present disclosure;

FIG. 24 is a perspective view of a second embodiment of a handpiece of an apparatus for laser treatment of the present disclosure;

FIG. 25 is a schematic diagram of a second embodiment of a handpiece of an apparatus for laser treatment of the present disclosure;

FIG. 26 is a perspective view of a third embodiment of a handpiece of an apparatus for laser treatment of the present disclosure;

FIG. 27 is a schematic diagram of a third embodiment of a handpiece of an apparatus for laser treatment of the present disclosure; and

FIG. 28 is a schematic diagram of a controller of an apparatus for laser treatment of the present disclosure.

### DETAILED DESCRIPTION

Hereinafter, preferred embodiments of the present disclosure will be described with reference to the attached drawings. However, the embodiments of the present disclosure may be modified in various other forms, and the scope of the present disclosure is not limited to the embodiments described below.

Additionally, the embodiments of the present disclosure are provided to more completely explain the present disclosure to those with average knowledge in the relevant technical field.

In the drawings, the shapes and sizes of elements may be exaggerated for a clearer explanation.

In describing embodiments of the present disclosure in detail, when it is determined that a detailed description of known technologies associated with the present disclosure may unnecessarily obscure the gist of the present disclosure, the detailed description thereof will be omitted. Furthermore, the terms described below are defined in consideration of functions in the present disclosure, and may vary according to the intention or practice of a user or an operator. Therefore, the definition thereof should be based on the content throughout this specification. The terms used in the description are intended to describe embodiments only, and shall by no means be restrictive. Unless clearly used otherwise, expressions in a singular form include a meaning of a plural form.

In the present description, an expression such as “comprising” or “including” is intended to designate characteristics, numbers, steps, operations, elements, a portion or combinations thereof, and shall not be construed to preclude any presence or possibility of one or more other characteristics, numbers, steps, operations, elements, parts or combinations thereof.

In this specification, the expressions such as “on,” “above,” “upper,” “below,” “beneath,” “lower,” and “side surface” are based on the drawings, and may actually vary depending on a direction in which elements or components are disposed.

Furthermore, throughout the specification, the terms “connected to” or “coupled to” are used to designate a connection or coupling of one element to another element and include both a case in which an element is “directly connected or coupled to” another element and a case in which an element is “indirectly connected or coupled to” another element via still another element.

As described below, the present disclosure will be described in detail through each aspect or each embodiment of the present disclosure, and each aspect or each embodiment described in the specification does not mean only one aspect or embodiment but also means a combination with other aspects or other embodiments. Accordingly, the citation of the claims in the scope of the patent claims is only one example and the technical idea of the present disclosure should not be interpreted only as a combination with the cited claims, and includes and various combinations with the claims are also included in the scope of the technical idea of the present disclosure.

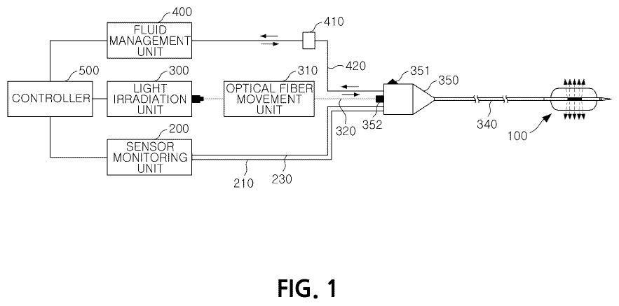

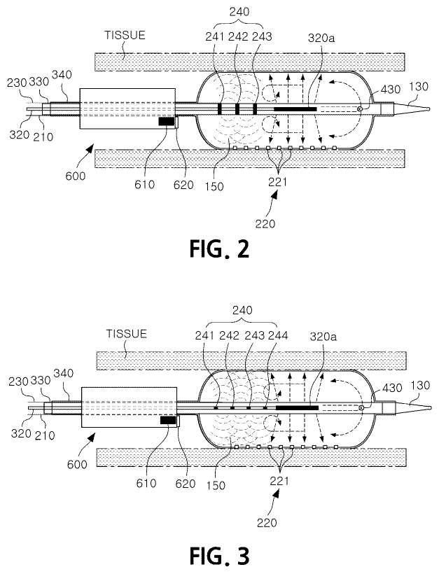

FIG. 1 is a schematic diagram of an apparatus for laser treatment according to a first embodiment of the present disclosure, and FIG. 2 is a schematic diagram of a catheter in an apparatus for laser treatment according to the first embodiment of the present disclosure.

An apparatus for laser treatment according to a first embodiment of the present disclosure may include a catheter 100, a sensor monitoring unit 200, a light irradiation unit 300, a fluid management unit 400, and a controller 500.

The light irradiation unit 300 may include a light source configured to irradiate light, and includes an optical fiber 320 connected to the light source to transmit light to a treatment site, an optical fiber movement unit 310, and a handpiece 350 for manipulating the optical fiber movement unit 310. The handpiece 350 includes a first manipulation portion 351 for manipulating forward/reverse movements of the optical fiber 320 and a second manipulation portion 352 for manipulating the rotation of the optical fiber 320. The configuration of the optical fiber movement unit 310, including the handpiece 350, will be described again below.

In the first embodiment, a catheter 100 is inserted, and the optical fiber 320 is manipulated by the handpiece 350, so that light from the light irradiation unit 300 is irradiated onto a tubular tissue within a body through the optical fiber 320 inserted into the catheter 100.

The optical fiber movement unit 310 controls a movement of the optical fiber 320 inserted into the catheter 100. The optical fiber movement unit 310 may control forward/backward movements and rotation of the optical fiber 320. Here, the optical fiber 320 within a balloon 150 may be moved manually or automatically using a transparent tube 330 as a rail, thereby allowing for wider and more selective treatment of lesions in the tubular tissue.

The optical fiber 320 may be comprised of a core, cladding, and a buffer for multimode operation, and the core may have a thickness of 0.2 mm to 1 mm. An optical fiber end 320a may be formed into a cylindrical shape and may irradiate uniform light, and the light may then pass through the transparent balloon 150 and may reach a target tissue.

The optical fiber end 320a may be protected by a high-transparency glass cap 323 (FIG. 17) and sealed with a high-melting-point epoxy. Furthermore, the glass cap 323 may be formed of materials such as quartz, pure silica, or polymethyl methacrylate (PMMA). The glass cap 323 is sealed with a rounded end to minimize interference during movement within the transparent tube 330 without damaging an inner surface of the transparent tube 330. The glass cap 323 will be described again in FIG. 17.

A delivery tube 340 connecting the catheter 100 and the handpiece may include a plurality of channels, and the plurality of channels include channels through which wires 210 and 230 connected to a plurality of sensors 221 and 240 pass, and channels for the transparent tube 330, the optical fiber 320 and a fluid tube 420.

The catheter 100 is provided with a flexible tip 130 on one end thereof and is guided and inserted into the tubular tissue, and the balloon 150 is inflated by the fluid to expand an internal tubular tissue. Since the balloon 150 expands and fixes a position within the tubular tissue, the balloon 150 may also be referred to as an expansion fixing portion.

An endoscope unit 600 disposed outside the delivery tube 340 and formed to surround at least a portion of the delivery tube 340 may be disposed on a rear end of the catheter 100 in an entry direction. The endoscope unit 600 includes a camera 610 disposed forward, i.e., toward the balloon 150, and a filter 620 disposed in front of the camera 610. Since any conventional endoscope is appliable to the endoscope unit 600, a detailed description thereof will be omitted in the present disclosure.

The catheter 100 is designed to be inserted into the endoscope channel in a Through-The-Scope (TTS) manner and may be configured to be smaller than an internal diameter of an insertion channel.

The catheter 100 may inject fluid into the balloon 150 once the catheter 100 reaches a desired position to uniformly expand an internal structure of a stenotic or narrowed tissue. Furthermore, the catheter 100 is provided with the flexible tip 130 to minimize mechanical damage to body tissue while entering the body. The flexible tip 130 may be formed of, for example, a block copolymer comprised of rigid polyamide blocks and flexible polyether blocks, polyurethane, silicone, rubber, or another material with a hardness between 10 and 60 Shore A.

The sensor monitoring unit 200 may receive information detected by a plurality of sensors provided in the catheter 100. The sensors include a piezoelectric sensor 240 which serves as a shape information sensor, a temperature sensor or pH sensor which serves as a tissue condition measurement sensor 221, and a tissue impedance measurement sensor.

A plurality of piezoelectric sensors 240 are spaced apart from each other by regular intervals in a length direction of the catheter 100 within the balloon 150. In this embodiment, the piezoelectric sensors 240 include first, second and third piezoelectric sensors 241, 242 and 243. A plurality of tissue condition measurement sensors 221 form a sensor array 220 and are disposed at the balloon 150, and upon inflation of the balloon 150, the tissue condition measurement sensors 221 are in contact with the tissue to obtain tissue condition information. Physical parameters of the tissue detected by the tissue condition measurement sensors 221 or the sensor array 220 are monitored.

The optical fiber 320 is provided in the catheter 100 to irradiate necessary light to the tissue.

The balloon 150 included in the catheter 100 may be formed of silicone, polyurethane, nylon, elastomer, or other thermoplastic elastic materials. Depending on the material, the pressure generated within the balloon may range from 0.1 to 100 psi. Specifically, at pressures ranging from 20 to 100 psi, the balloon 150 may be formed of materials such as acrylic, polyethylene terephthalate (PET), and nylon, which may expand to a small expansion range of 0 to 10%. Furthermore, in the case of the balloon 150 formed of silicone, polyurethane, or nylon elastomers, pressure ranging from 0.1 to 5 psi may be applied thereto, so that the balloon 150 may expand to a large expansion range of 10 to 200%. A shape of the expanded balloon 150 may vary depending on the type of tubular tissue.

The controller 500 may receive measurement information from the sensor monitoring unit 200 and may control the light source of the light irradiation unit 300.

For example, the controller 500 may be implemented by a processor, program instructions executed by the processor, a software module, microcode, a computer program product, a logic circuit, an application-specific integrated circuit, firmware, or the like.

The fluid management unit 400 supplies fluid to inflate the balloon 150 of the catheter 100 and removes the supplied fluid. The fluid management unit 400 may be connected to the controller 500 and may be controlled by the controller 500.

The fluid injected into the balloon 150 by the fluid management unit 400 is not particularly limited and may be a gas such as air or a liquid such as distilled water, saline solution, deionized water, or other contrast agents.

The fluid management unit 400 may include an air trap remover 410 capable of removing an air trap within the balloon 150 and a cooler (not illustrated) generating cooling fluid for lowering a temperature of the fluid.

When the fluid injected by the fluid management unit 400 is a liquid, an air trap may be formed inside the balloon 100 or in the fluid tube 420 which is a portion of the fluid management unit 400 and through which the fluid is injected, and thus, the air trap remover 410 may be included to remove the air trap.

The air trap remover 410 minimizes adverse effects caused by air trapped inside the balloon 150 during the procedure. The air trap inside the balloon 150 may uncontrollably alter the light distribution irradiated by the optical fiber 320, and the air trap remover 410 may be provided to address this issue.

An outlet 430 is disposed in an end of the fluid tube 420, and the outlet 430 is disposed closer to the flexible tip 130 than the optical fiber end 320a to deliver the fluid injected by the fluid management unit 400 into the balloon 150.

Before injecting liquid into the catheter 100, bubbles representing air traps may be detected in advance using other technologies such as an infrared radiation detector or ultrasound imaging and may be removed.

Additionally, a cooler prevents tissue damage by cooling the tissue, particularly the mucosa or submucosal membrane. For example, cooling irrigation via a pump or an inflator may be included. The fluid flow and temperature within the balloon 150 may be measured by the sensor 221, and the temperature and flow rate may be transmitted to a monitoring device and may be controlled by the controller 500. Specifically, the controller 500 may control the supply, flow, or temperature of the fluid supplied and aspirated from the fluid management unit 400. Furthermore, the fluid management unit 400 may further include a fluid tube 420 for delivering the fluid.

When the sensor 221 or the sensor array 220 is attached to a portion of the catheter 100, physical parameters of the tissue may be detected. For example, physical parameters may include tissue stress-strain, pH level, and mucosal impedance of the mucosal surface. The treatment process may be monitored through these physical parameters. However, the sensor 221 or the sensor array 220 is not limited to the detection of these physical parameters, and may detect unwanted events occurring in a patient during treatment by recording an electrical signal from the nervous system using single or multiple sensors 221.

The piezoelectric sensor 240 may be disposed inside the balloon 150 and may be located in a central portion when viewed longitudinally, and may process an impedance signal and transmits the signal to the controller 500, thereby monitoring the pressure of the balloon 150 and a distance between the balloon 150 and the tissue, i.e., a diameter of the balloon 150, and allowing for optimization of the treatment dose based on the tissue/balloon diameter.

Furthermore, since air bubbles representing air traps cause changes in the impedance signal within the inflated balloon 150, so that the piezoelectric sensor 240 may be monitored to detect air traps in advance.

The piezoelectric sensor 240 is disposed as being disposed inside the transparent tube 330 in FIG. 2. As illustrated in FIG. 2, impedance waves are disposed within the balloon 150 of the catheter 100. The piezoelectric sensor 240 is cylindrical and may be formed of piezoelectric ceramic, single-crystal material, or thin-film piezoelectric material. The piezoelectric sensor 240 may range in size from 1 to 3 mm in diameter and 1 to 5 mm in length, and to prevent the piezoelectric sensor 240 from absorbing light, the piezoelectric sensor 240 is disposed adjacently to the beginning of the balloon 150 to minimize a path for light to reach the piezoelectric sensor 240.

The sensor monitoring unit 200 may estimate a diameter of the balloon 150 from the monitored pressure of the balloon 150, and may select and control the optimal dosage, and if necessary, the treatment dose may be adjusted based on the measured temperature, and a target temperature may be controlled to maintain 45 to 65° C., thereby protecting the tissue mucosa and submucosal layer from overheating. Furthermore, the sensor monitoring unit 200 may define the level of gastric acid based on various physical parameters, including a measured pH level, or may analyze the impedance of the tissue and mucosa and an electrical signal from the nerves to control the effects on the nervous system, and for safety, a forced control mode may be provided to terminate treatment when the temperature or strain exceeds a threshold.

According to an embodiment of the present disclosure, the optical fiber 320, the wires 210 and 230, the fluid tube 420, and the optical fiber movement unit 310 may be included. The wires 210 and 230, the optical fiber 320 and the fluid tube 420 may be integrated into an inner lumen of the delivery tube 340.

FIG. 3 illustrates a schematic diagram of a catheter in an apparatus for laser treatment according to a modified embodiment of the first embodiment of the present disclosure. The catheter 100 illustrated in FIG. 3 is identical to the catheter 100 illustrated in FIG. 2 except for the piezoelectric sensor 240, and thus, the description of the remaining parts, excluding the piezoelectric sensor 240, will be replaced with the description of FIG. 2.

In the catheter 100 illustrated in FIG. 3, the piezoelectric sensor 240 is disposed within the balloon 150. Here, the piezoelectric sensor 240 is not disposed within the transparent tube 330, but rather within the optical fiber 320. The impedance waves within the catheter 100 are indicated by a curved arrow along the optical fiber 320. Additionally, in the catheter 100 of FIG. 3, the piezoelectric sensor 240 includes first to fourth piezoelectric sensors 241 to 244. In the present disclosure, the number of piezoelectric sensors 240 may vary depending on the length of the balloon 150 and a distance by which the optical fiber 320 moves within the balloon 150.

When the piezoelectric sensor 240 is attached to the optical fiber 320 as illustrated in FIG. 3, a position of the optical fiber end 320a and a position of the piezoelectric sensor 240 may be interlinked with each other, and the position of the piezoelectric sensor 240 changes according to the movement of the optical fiber 320, thereby accurately obtaining shape information of the tissue to be treated.

In the first embodiment of the present disclosure, internal shape information of tubular tissue may be obtained through the piezoelectric sensor 240, through which the controller 500 may be manipulated to perform accurate treatment. Specifically, diameters of tubular tissues are different from person to person, and for accurate treatment, it is necessary for the balloon 150 to be settled in the tubular tissue so that a position thereof is secured therein. Accurate treatment may be possible by obtaining tubular tissue shape information, such as the diameter, through a shape information sensor, and supplying fluid through the fluid management unit 400 to adjust the expansion/contraction of the balloon 150 accordingly.

Additionally, in the first embodiment, the shape information of the tubular tissue is obtained by measuring impedance using a piezoelectric sensor 240 and calculating the diameter/pressure accordingly, but the present disclosure is not limited to this method, and other methods for obtaining shape information may be applied thereto. However, the present disclosure utilizes the catheter 100 in a TSS method, and preferably, a size thereof may be large enough to be inserted into an endoscopic channel. Alternatively, the piezoelectric sensor 240 may be used for ultrasound oscillation/ultrasonic measurement to obtain the diameter (shape information).

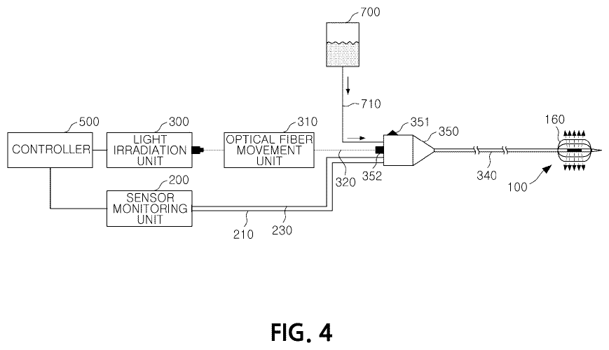

FIG. 4 illustrates a schematic diagram of an apparatus for laser treatment according to a second embodiment of the present disclosure.

The apparatus for laser treatment according to a second embodiment of the present disclosure includes a catheter 100, a sensor monitoring unit 200, a light irradiation unit 300, a fluid storage unit 700, and a controller 500.

The light irradiation unit 300 may include a light source configured to irradiate light, and includes an optical fiber 320 connected to the light source to transmit light to the treatment site, an optical fiber movement unit 310, and a handpiece 350 for manipulating the optical fiber movement unit 310. The handpiece 350 includes a first manipulation portion 351 for manipulating forward/reverse movements of the optical fiber 320 and a second manipulation portion 352 for manipulating the rotation of the optical fiber 320. The optical fiber movement unit 310 controls the movement of the optical fiber 320 inserted into the catheter 100.

The delivery tube 340 connecting the catheter 100 and the handpiece 350 may include a plurality of channels, and the plurality of channels include channels through which wires 210 and 230 connected to a plurality of sensors 221 and 240 (see FIG. 2) pass, and channels for a transparent tube, an optical fiber 320, and a fluid tube 710.

The catheter 100 includes a wire basket 160, and the optical fiber 320 is disposed within the wire basket 160. The wire basket 160 has a structure for uniformly expanding an internal structure of stenotic or narrowed tissue, and when the wire basket 160 reaches a desired position, the wire basket is pushed and expands radially, in which case an optical fiber end is disposed in a central portion of the tubular tissue. The wire basket 160, similarly to the balloon 150, expands and secures a position thereof within the tubular tissue, and may thus be referred to as an expansion fixing portion.

The wire basket 160 is formed by coupling several basket wires formed of a deformable shape-memory alloy. The basket wire may be used to form a contractile device for expanding the tissue of a tubular organ. The wire may be formed of metals, including shape-memory alloys such as nickel-titanium alloy (nitinol), titanium-palladium-nickel, nickel-titanium-copper, gold-cadmium, iron-zinc-copper-aluminum, and titanium-niobium-aluminum. The shape of the wire basket 160 will be described below.

The sensor monitoring device 200 includes a piezoelectric sensor 240 (see FIG. 2) as a shape information sensor, a plurality of sensors 221 (see FIG. 2) as tissue condition measurement sensors, and wires 210 and 230 connected to the shape information sensors and the tissue condition measurement sensor.

The controller 500 may receive measurement information from the sensor monitoring device 200 and may control the light irradiation unit 300.

The fluid reservoir 700 is configured to supply liquid to cool the optical fiber end 320a (see FIG. 2) disposed within the catheter 100, and prevents thermal damage to tissue when a high-power laser is used as a light source. The amount of cooling fluid may be controlled via a peristaltic pump, but the cooling fluid may also be supplied through gravity.

When a sensor is attached to the basket wire of the wire basket 160, the sensor may detect physical parameters of the tissue. For example, physical parameters may include temperature, tissue stress-strain, a pH level, and impedance of the mucosal surface. Through the physical parameters, a treatment process may be monitored. However, detection is not limited only to physical parameters, and electrical signals from a nervous system may also be recorded via one or more sensors, thereby allowing for the detection of unwanted events that may occur in the patient during treatment.

The sensor monitoring device may adjust the treatment dose based on a measured temperature, and limit a target temperature to 60 to 80° C., thereby increasing the safety of the treatment procedure and protecting deeper tissue layers (e.g., muscle layers) from overheating.

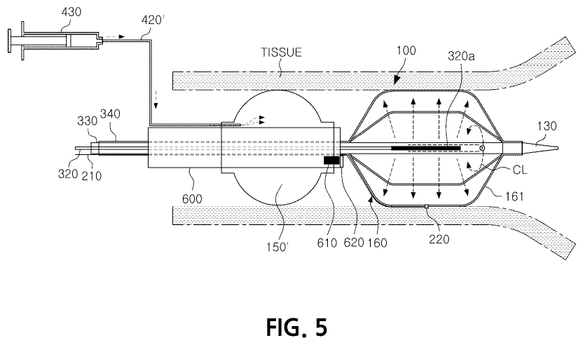

FIG. 5 illustrates a schematic diagram of a catheter in an apparatus for laser treatment according to a third embodiment of the present disclosure.

The apparatus for laser treatment according to the third embodiment includes a catheter 100, a light irradiation unit, a sensor monitoring unit, and a controller, and the configurations of the light irradiation unit, the sensor monitoring unit, and the controller are not significantly different from those of the first and second embodiments, and will therefore be briefly described. Unlike the first and second embodiments, the catheter 100 in the third embodiment includes a balloon 150′ and a wire basket 160.

The balloon 150′ and wire basket 160 is disposed in series, and the wire basket 160 is disposed close to the flexible tip 130, and the balloon 150′ is disposed behind the wire basket 160. The balloon 160 may be disposed at the endoscope unit 600.

The optical fiber 320 may pass through the balloon 150′ and may extend to locate the optical fiber end 320a within the wire basket 160, and the optical fiber 320 may be moved forward/backward and rotated within the wire basket 160.

Furthermore, the flexible tip 130 disposed on the front end of the wire basket 160 is disposed to assist in guiding and inserting the optical fiber into tubular tissue. The flexible tip 130 is formed of a flexible material to prevent tissue damage when the wire basket 160 enters the tissue.

The sensor array 220 may be provided on an outer surface of the wire basket 160 to form a sensor array. Specifically, a temperature sensor and a pressure sensor are attached to specific positions on an outer surface of a basket wire 161 to form a contraction and ensure treatment safety. Both the temperature sensor and the pressure sensor are attached to the outer surface of the basket wire 161 to provide information on the temperature and the pressure of the tubular organ tissue.

Furthermore, the transparent tube 330 is additionally provided within the catheter 100 and penetrates through the delivery tube 340, and is disposed in one of the channels of the delivery tube 340. A wire 210 connected to the sensor array 220 or the sensor 221 is disposed in another channel of the delivery tube 340. The optical fiber 320 is disposed integrally with the transparent tube 330 and may be moved.

Information detected by sensors attached to the basket wires 161 of the wire basket 160 may be received. The sensors may be provided through a single or multiple sensor array, and monitor physical parameters of the tubular tissue detected by the sensor or sensor array.

The catheter 100 including the wire basket 160 is designed in a through-the-scope (TTS) manner to be inserted into an endoscopic channel, and an inner diameter of the insertion channel may range from 2.8 mm to 4.3 mm. The optical fiber 320 is connected to a laser light source, and light is irradiated from an optical fiber end 320a.

The optical fiber end 320a has an effective length of 0.5 mm to 20 mm, depending on the length of the target tissue, and may irradiate light. The optical fiber end 320a may be formed in a cylindrical shape to irradiate uniform light, and the light passes through a small, thin basket wire 161 and reaches a mucosal surface of the tubular tissue.

The optical fiber end 320a may be protected by a high-transparency glass cap 323 (see FIG. 17) and may be sealed with a high-melting-point epoxy. Materials for the glass cap may include quartz, pure silica, and polymethyl methacrylate (PMMA).

Furthermore, the endoscope unit 600 may be equipped with a filter 620 to protect the camera 610 and the image sensor of the camera 610, through which the entire procedure and treatment site may be clearly visualized. The filter may be formed as a thin film having a thickness of 100 μm to 1 mm.

The balloon 150′ may be disposed in the endoscope unit 600, and fluid may be supplied through the external syringe 430. The syringe 430 may be connected to the balloon 150′ via a fluid tube 420′, and the fluid tube 420′ may be inserted together into the delivery tube 340, or may be attached to the endoscope unit 600 and inserted together with the delivery tube 340 from outside the delivery tube 340.

The balloon 150′ may be formed of materials such as urethane, silicone rubber, or pelletane, and hardness thereof ranges from 90 Shore A to 50 Shore A. During use thereof, the balloon 150′ may be inflated at a pressure of 0.2 to 5 PSI. The lower hardness material may inflate the balloon from 12 mm to 30 mm to support the wire basket in the central portion of the tubular tissue in accordance with various tubular diameters. This may aid in delivering light energy to the tubular tissue through a cylindrical light distribution.

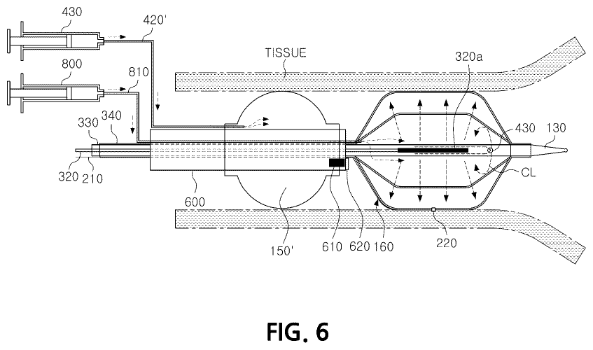

FIG. 6 illustrates a schematic diagram of a catheter in a phototherapy device according to a fourth embodiment of the present disclosure.

The fourth embodiment of FIG. 6 includes the same configuration as the third embodiment of FIG. 5, and, in addition to the third embodiment, the fourth embodiment includes a fluid injection channel 810 capable of injecting fluid to remove contaminants from the wire basket 160 or the transparent tube 330.

Specifically, the fluid injection channel 810 is connected to a fluid supply portion 800, such as a syringe, and is connected to the delivery tube 340, and extends to the wire basket 160. Accordingly, the fluid injection channel 810 extends by a starting point of the wire basket 160 and provides fluid toward the wire basket 160 or the transparent tube 330 before expansion, thereby cleaning the wire basket 160 or the transparent tube 330.

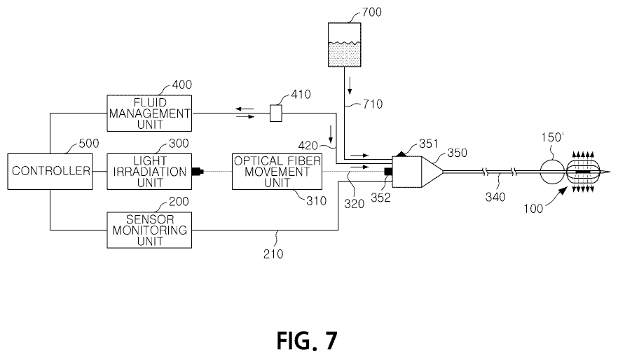

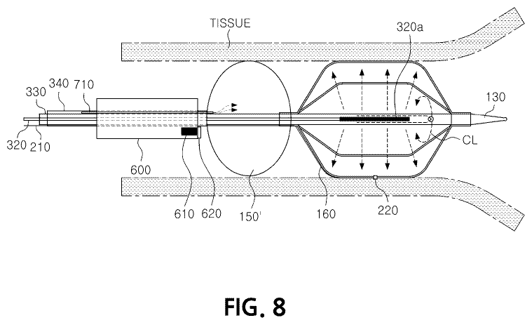

FIG. 7 illustrates a schematic diagram of a phototherapy device according to a fifth embodiment of the present disclosure, and FIG. 8 illustrates a schematic diagram of a catheter of the phototherapy device according to the fifth embodiment.

An apparatus for laser treatment according to a fifth embodiment of the present disclosure includes a catheter 100, a sensor monitoring unit 200, a light irradiation unit 300, a fluid management unit 400, a fluid storage unit 700, and a controller 500.

The catheter 100 includes a balloon 150′ and a wire basket 160.

The balloon 150′ and the wire basket 160 are disposed in series, the wire basket 160 is disposed close to the flexible tip 130, and the balloon 150′ is disposed behind the wire basket 160. The balloon 160 may be disposed at the endoscope unit 600.

The optical fiber 320 may pass through the balloon 150′ and may extend to be located on the optical fiber end 320a within the wire basket 160, and the optical fiber 320 may be moved forward/backward and rotated within the wire basket 160.

Furthermore, a flexible tip 130 disposed on a front end of the wire basket 160 is disposed to assist in guiding and inserting the optical fiber into tubular tissue. The flexible tip 130 is formed of a flexible material to prevent tissue damage when the wire basket 160 enters the tissue.

The sensor array 220 may be provided on an outer side surface of the wire basket 160.

Furthermore, the transparent tube 330 is additionally provided within the catheter 100, penetrates through the delivery tube 340, and is disposed in one of the channels of the delivery tube 340. The wire 210 connected to the sensor array 220 is disposed in another channel of the delivery tube 340. The optical fiber 320 is disposed integrally with the transparent tube 330 and is movable.

The fluid tube 420 connected to a fluid management unit 400 is disposed inside the delivery tube 340. An outlet 430 is disposed on the end of the fluid tube 420, and the outlet 430 is disposed closer to the flexible tip 130 than the optical fiber end 320a to deliver the fluid injected by the fluid management unit 400 into the wire basket 160. The fluid discharged from the wire basket 160 may cool a surrounding tissue, and may thus prevent the tissue from overheating due to light irradiation. Since the fluid used for cooling is not recovered but instead enters the human body, a fluid that is harmless to the human body is used.

Meanwhile, the fifth embodiment includes a fluid storage unit 700 for inflating/deflating the balloon 150′. The fluid in the fluid storage unit 700 is connected to the balloon 150′ via the fluid tube 710. The fluid storage unit 700 may be supplied by a pump (not illustrated) or gravity. In the present disclosure, the fluid management unit 400 and the fluid storage unit 700 may perform a cooling function or inflate/deflate the balloons 150 and 150′ depending on the supplied fluid and the discharge position.

In the fifth embodiment, the inflation/deflation of the balloon 150′ is not controlled by the controller 500, but the fluid management unit 400 for cooling is controlled by the controller 500. Accordingly, the controller 500 may manipulate the optical fiber 320 or may control the fluid management unit 400 based on the information obtained by the sensor array 220.

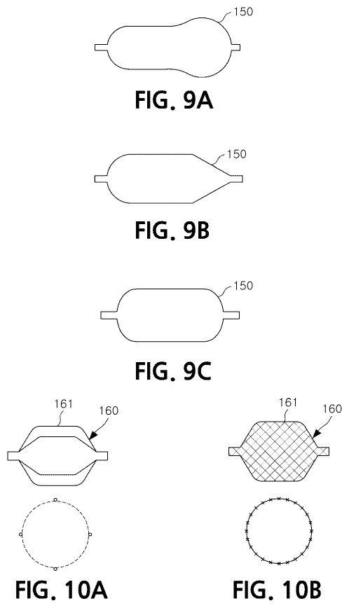

FIGS. 9A and 9B illustrate various shapes of balloons 150. The balloon 150 may take on various shapes other than those illustrated in FIGS. 1 to 8. FIG. 9A illustrates a pear-shaped balloon 150 in which one end thereof is expanded, FIG. 9B illustrates a balloon 150 having a conical end, and FIG. 9C illustrates a cylindrical balloon 150. The shape of the balloon 150 may vary depending on the target tubular tissue and application field.

In the case of FIG. 9A, a balloon 150 for BE treatment is advantageous for contact with tubular sphincter tissue and the gastroesophageal junction (GE junction). The pear-shaped balloon 150 in which one end thereof is expanded assists in fixing the balloon 150 to body tissue and is suitable for treating tissues of various diameters.

In the cases of FIGS. 9B and 9C, the balloon has a spherical shape on a proximal end thereof, which makes it advantageous for visualizing treatment results through an endoscopic camera. Furthermore, in the cases of FIGS. 9B and 9C, this is advantageous for introduction into an internal channel of the endoscope after treatment. Depending on the tubular tissue used, an expanded diameter of the balloon 150 may range from 12 to 30 mm, and a length thereof may range from 15 to 200 mm.

FIGS. 10A and 10B illustrate schematic diagrams of the wire basket.

As illustrated in FIG. 10A, the wire basket 160 may be comprised of at least four basket wires 161. A plurality of wires are arranged to be spaced apart from each other by regular intervals on an outer surface of the optical diffusion optical fiber end 320a (see FIG. 8).

As illustrated in FIG. 10B, the wire basket 160 may also be configured so that the basket wires 161 may be formed in a mesh shape.

To minimize the thermal effect of irradiated light and simultaneously minimize energy absorption by the basket wires 161, the material of the basket wires 161 may preferably have a high reflectivity (>95% at wavelengths of 400 to 3000 nm) for the wavelength of the irradiated light.

To ensure that the basket wires 161 maintain a high reflectivity when electromagnetic energy is emitted, the basket wires 161 may be coated with gold or silver. Additionally, an energy irradiation method may be adjusted for sequential treatment with short irradiation times to minimize a temperature increase in the basket wire 161.

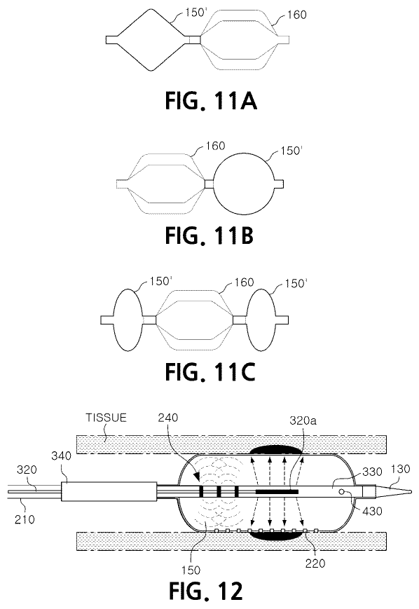

FIGS. 11A to 11C illustrate various configurations of catheters including a balloon-wire basket.

As in the fifth embodiment, the present disclosure may simultaneously include a balloon 150′ and a wire basket 160. In this case, light irradiation is performed in the wire basket 160, and the balloon 150′ serves to fix the catheter 100 in the tubular tissue.

A balloon-wire basket structure has the following advantages:
1) A basket-integrated optical fiber provides laser light that may directly heat the mucosa/submucosa without natural cooling by liquid, thereby enhancing selective treatment of the mucosa.
2) The balloon 150′ locates a basket-integrated optical fiber component in the central portion of the tubular tissue and supports the same. As compared to the optical fiber disposed in the balloon 150, the optical fiber disposed in the balloon 150′-basket 160 may minimize the thickness reduction of the mucosa/submucosa due to a weak structure of the basket wire 161. Accordingly, selective resection/removal of the mucosa/partial submucosa may be performed, with no or minimal thermal damage to a muscle layer.

The shape of the inflated balloon 150′ may be formed into a square, circular, oval, conical, tapered, or stepped shape, depending on the type of tubular tissue.

With respect to an arrangement of the balloon 150′ and the wire basket 160, as illustrated in FIG. 11A, the wire basket 160 may be disposed in front of the balloon 150′, as illustrated in FIG. 11B, the wire basket 160 may be disposed in back of the balloon 150′, and as illustrated in FIG. 11cC the balloons 150′ may be disposed in front and back of the wire basket 160.

Here, “front” and “back” refer to the front when a position is close to a distal end of the catheter, i.e., the flexible tip 130 (see FIG. 2), and the back when the position is farther away.

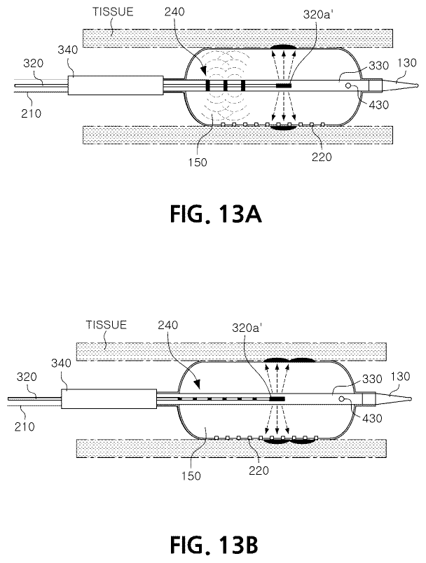

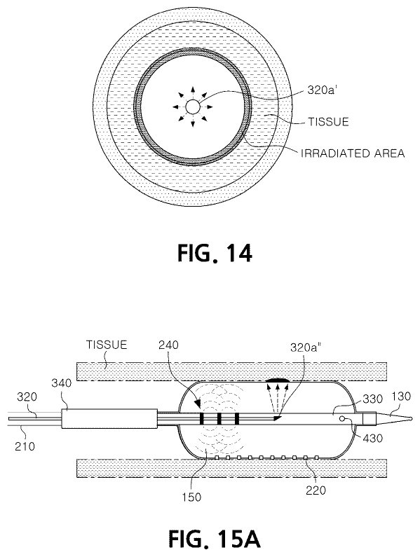

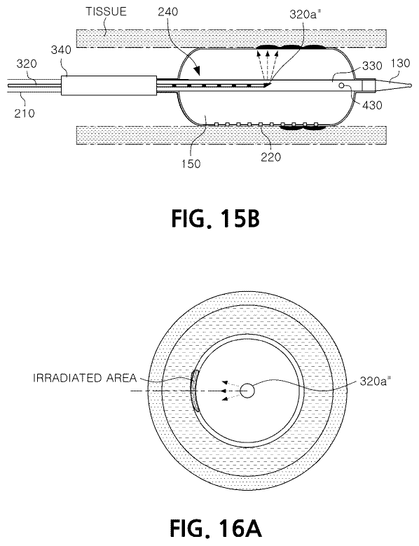

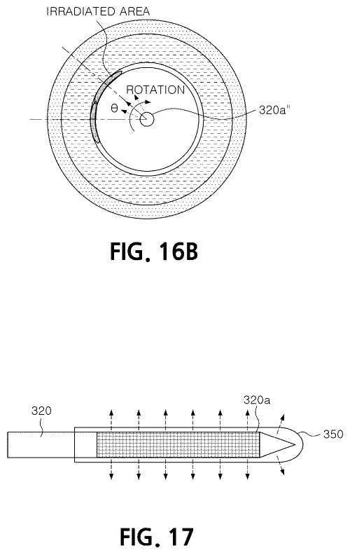

FIGS. 12 to 16B illustrate schematic diagrams of catheters according to sixth to eighth embodiments of the present disclosure. Specifically, FIG. 12 illustrates a schematic diagram of a catheter according to the sixth embodiment of the present disclosure. FIG. 13A is a schematic diagram of a catheter according to the seventh embodiment of the present disclosure, FIG. 13B is a schematic diagram of a modified embodiment of the seventh embodiment, and FIG. 14 is a schematic cross-sectional diagram of the catheter according to the seventh embodiment. FIG. 15A is a schematic diagram of a catheter according to the eighth embodiment of the present disclosure, FIG. 15B is a schematic diagram of a modification of the eighth embodiment, and FIGS. 16A and 16B are schematic cross-sectional diagrams of the eighth embodiment of the present disclosure.

The basic structures of the sixth, seventh, and eighth embodiments are identical, and the sixth, seventh, and eighth embodiments differ only in the type of optical fiber 320. The sixth embodiment includes a diffusion optical fiber, the seventh embodiment includes a radial optical fiber, and the eighth embodiment includes a side-firing fiber.

The common basic structures of the sixth to eighth embodiments are identical to those of the first embodiment. That is, the apparatus for laser treatment of the sixth to eighth embodiments also have a similar structure to the first embodiment, including a catheter 100, the sensor monitoring unit 200 (see FIG. 1), the light irradiation unit 300 (See FIG. 1), the fluid management unit 400 (see FIG. 1), and the controller 500 (see FIG. 1), and thus, detailed descriptions thereof will be omitted.

In the sixth embodiment illustrated in FIG. 12, the optical fiber 320 includes a diffusion-type optical fiber, and light is emitted from the optical fiber end 320a to a surrounding area, thereby allowing for a relatively long treatment range of 10 to 30 mm. In the case of a diffusion-type optical fiber, light is dispersed throughout the entire tubular tissue at a long length.

In the seventh embodiment illustrated in FIGS. 13A, 13B and 14, the optical fiber 320 includes a radial optical fiber, and may have a relatively short treatment range of 5 to 7 mm in an optical fiber end 320a′. The radial optical fiber of the seventh embodiment provides a shorter distance in the longitudinal direction, more focused light than in the sixth embodiment, but is identical to the sixth embodiment in that the light is distributed within the tubular tissue.

In the seventh embodiment, the piezoelectric sensor 240 is disposed in the transparent tube 330, and in a modified embodiment of the seventh embodiment, the piezoelectric sensor 240 is attached to the optical fiber 320. When the piezoelectric sensor 240 is disposed in the transparent tube 330, the piezoelectric sensor 240 provides shape information on the tubular tissue in a fixed position, regardless of the forward or reverse movement/rotation of the optical fiber 320, but as in the modified embodiment, when the piezoelectric sensor 240 is attached to the optical fiber 320, the piezoelectric sensor 240 may move with the optical fiber 320 and may provide shape information in various positions, thereby enabling more precise treatment.

In the eighth embodiment illustrated in FIGS. 15A 15B and 16, the optical fiber 320 may include a side-firing fiber, and may have a relatively short treatment range of 5-7 mm from the optical fiber end 320a″, and in the case of the side-firing fiber, light is focused and irradiated on a portion of the tubular tissue. Since the side-firing fiber irradiates light in a partial direction and at a partial length of the tubular tissue, localized treatment may be performed, and treatment of a wider area may also be achieved by moving the side-firing fiber. Accordingly, the flexibility of the treatment procedure may be improved.

In the eighth embodiment (FIG. 15A), the piezoelectric sensor 240 is disposed on the transparent tube 330, while in a modified embodiment of the eighth embodiment (FIG. 15B), the piezoelectric sensor 240 is attached to the optical fiber 320. Specifically, a direction in which the piezoelectric sensor 240 measures a diameter may be interlined with a direction in which the side-firing fiber irradiates light, so that control via the controller 500 (see FIG. 1) may be more precise.

Even if the optical fiber 320 is disposed in the central portion of the tubular tissue, the tubular tissue may not be in a circular shape, and thus, the range of light irradiated by the side-firing fiber may vary depending on the orientation. When the piezoelectric sensor 240 is attached to the optical fiber 320 and rotates therewith, changes in diameter may be detected through the piezoelectric sensor 240, and based thereon, the controller 500 (see FIG. 1) may adjust other factors, such as the amount of light irradiated, a cooling liquid, and a balloon size, thereby providing the amount of light irradiated, required to the tissue.

As illustrated in FIGS. 16A and 16B, after light irradiation is completed on a specific area, the optical fiber 320 rotates to irradiate adjacent tissues, enabling continuous light irradiation across the irradiated area.

When the balloon 150 expands, a thickness of the submucosal layer decreases to 1 to 3 mm, and a uniform layer may be formed around a surface of the balloon 150, and depending on a target tissue layer, a wavelength appropriate for thermal penetration is selected and irradiated. In the present disclosure, wavelengths of 405, 490, 532, 585, 755, 980, 1470, 1550, and 2200 nm may be used for thermal treatment of the surface, ablation, removal, destruction, or coagulation thicknesses of 0.5 to 2 mm may be achieved. In this case, a radiation exposure range may be 0.1 to 1 kJ/cm², and the power range may be 5 to 100 W.

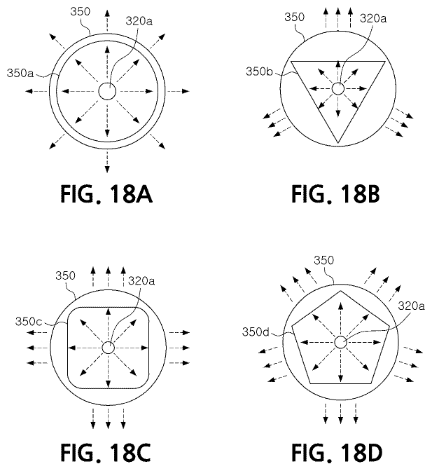

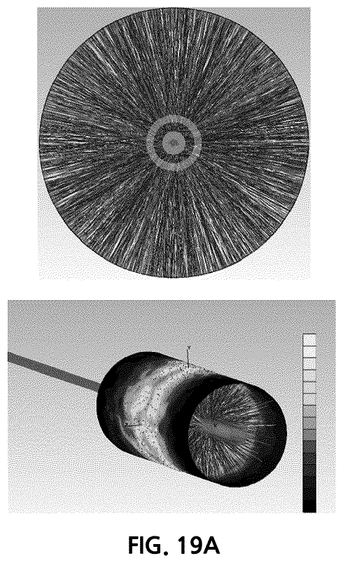

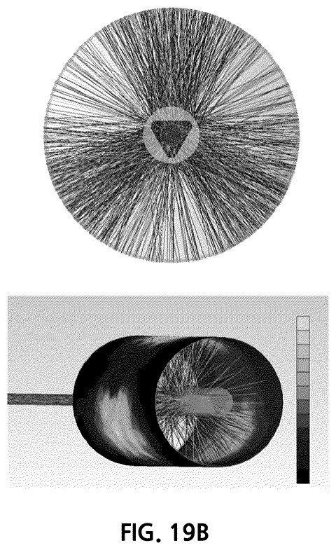

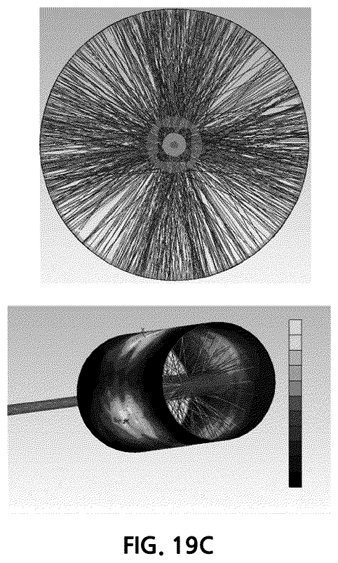

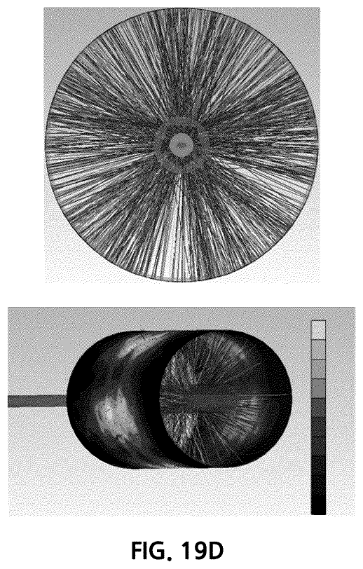

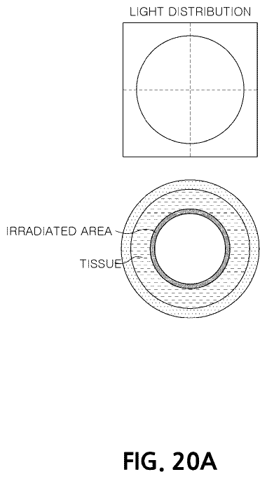

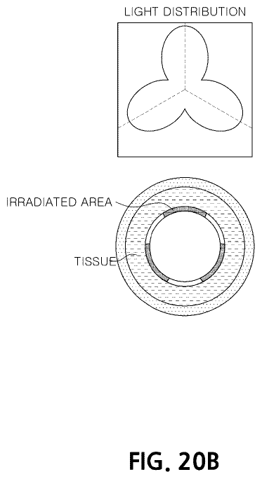

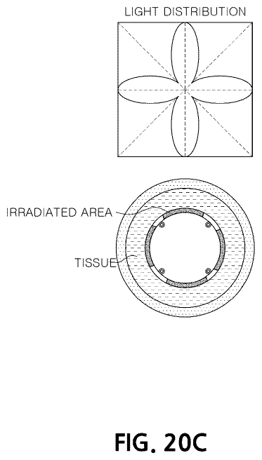

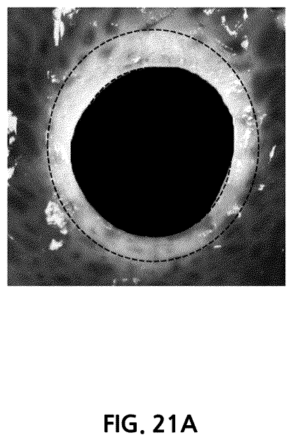

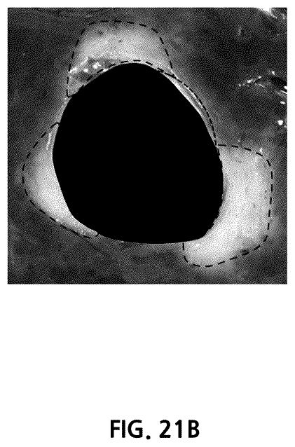

FIGS. 17 to 21B illustrate the adjustment of the light irradiation range according to the optical fiber end and the glass cap of the present disclosure. Specifically, FIG. 17 schematically illustrates the optical fiber end of the present disclosure, and FIGS. 18A to 18D schematically illustrate light irradiation according to changes in the glass cap of the optical fiber end. FIGS. 19A to 19D illustrate views of simulating light irradiation according to changes in the glass cap of the optical fiber end, corresponding to FIGS. 18A to 18D. FIGS. 20A to 20C are schematic diagrams illustrating tissues processed according to changes in the glass cap on the end of an optical fiber, corresponding to FIGS. 18A to 18C. FIGS. 21A and 21D are photographs illustrating tissues processed according to changes in the glass cap on the end of an optical fiber, corresponding to FIGS. 18A and 18B.

As illustrated in FIG. 17, in the present disclosure, the optical fiber 320 includes an optical fiber end 320a, and the glass cap 323 is covered on the optical fiber end 320a. As described above with reference to FIGS. 12 to 16B, the change in the light irradiated from the optical fiber end 320a depending on the type of optical fiber is explained. The optical fiber end 320a may have a conical diffuser tip and an embossed shape, and in the glass cap 323, an inner surface 323a may have various shapes when viewed in across-section, perpendicular to a longitudinal direction, thereby altering a light irradiation pattern.

The optical fiber end 320a may be protected by the highly transparent glass cap 323 and may be sealed with a high-melting point epoxy. Materials for the glass cap 323 may include quartz, pure silica, and polymethyl methacrylate (PMMA). The glass cap 323 is designed so that an end thereof is sealed in a rounded shape to prevent damage to the optical fiber when the optical fiber moves within the transparent tube 330 (see FIG. 2). The glass cap 323 has a length of 5 to 30 mm and an outer diameter of 0.8 to 3 mm, so that the glass cap 323 may be inserted into the internal channel (ID=2.8 to 4.3 mm) of an endoscope.

FIG. 18A illustrates a case in which the glass cap 323 has a circular inner surface 323a. As illustrated in FIGS. 18A, 19A, 20A and 21A, when the glass cap 323 has a circular inner surface 323a, light irradiated from the optical fiber end 320a is uniformly irradiated in the entire circumferential direction.

As illustrated in FIGS. 18B, 19B, 20B and 21B, when the glass cap 323 has a triangular inner surface 323a, light irradiated from the optical fiber end 320a is focused and irradiated in three directions along the triangular inner surface.

As illustrated in FIGS. 18C, 19C and 20C, when the glass cap 323 has a rectangular inner surface 323a, the light irradiated from the optical fiber end 320a is focused and irradiated in four directions along the rectangular inner surface.

As illustrated in FIGS. 18D and 19D, when the glass cap 323 has a pentagonal inner surface 323a, the light irradiated from the optical fiber end 320a is focused and irradiated in four directions along the pentagonal inner surface.

The partial angular light distribution may selectively ablate or coagulate the mucosa/submucosa, and may minimize thermal damage to the entire tubular tissue, thereby reducing the risk of post-treatment stenosis.

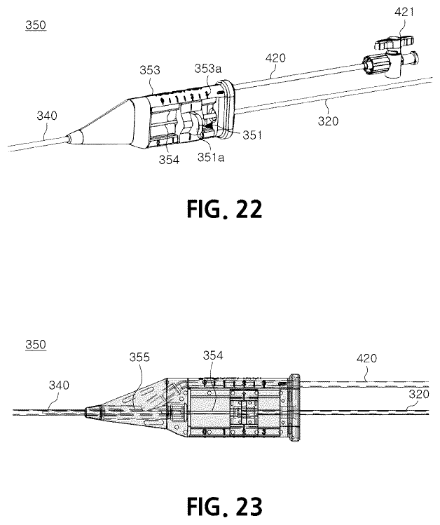

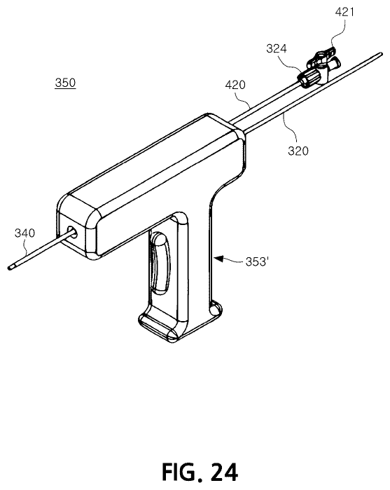

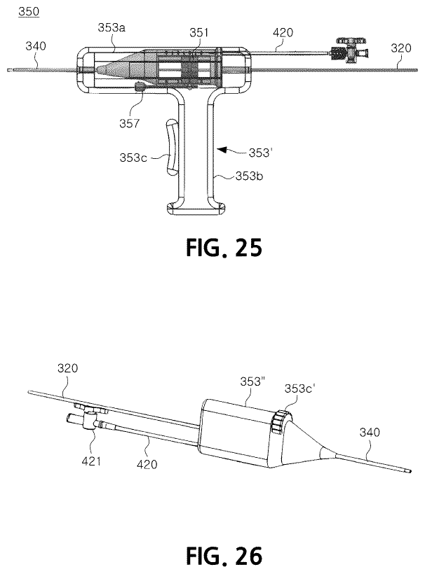

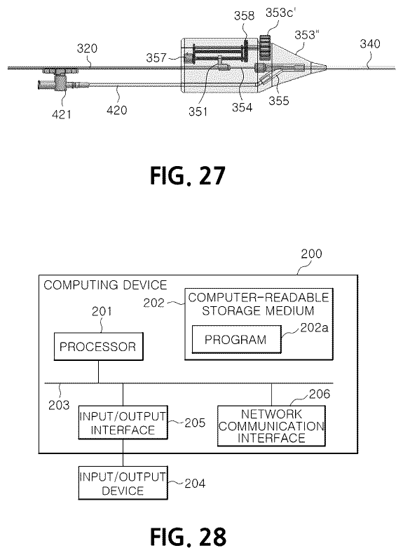

FIGS. 22 to 27 illustrate a handpiece of an apparatus for laser treatment of the present disclosure. Specifically, FIG. 22 is a perspective view of a first embodiment of a handpiece of an apparatus for laser treatment of the present disclosure, and FIG. 23 is a schematic diagram of the first embodiment of the handpiece of the apparatus for laser treatment of the present disclosure. FIGS. 24 and 25 are perspective views and schematic diagrams of a second embodiment of a handpiece of the apparatus for laser treatment of the present disclosure. FIGS. 26 and 27 are perspective views and schematic diagrams of a third embodiment of a handpiece of the apparatus for laser treatment of the present disclosure.

The first embodiment of the handpiece 350 illustrated in FIGS. 22 and 23 has a structure in which the optical fiber 320 is manually moved.

In the handpiece 350, the fluid tube 420 and the optical fiber 320 are introduced and pass through a Y-shaped connector 355 and are received in an internal channel of a delivery tube 340.

The handpiece 350 includes a handpiece cover 353, a support needle 354, and a first manipulation portion 351. The handpiece cover 353 forms an outer shape, and the Y-shaped connector 355 is formed inside, into which the fluid tube 420 and the optical fiber 320 are introduced and combined into the delivery tube 340. The optical fiber 320 passes through a central portion of the handpiece cover 353, and the support needle 354 is disposed to support the passing optical fiber 320.

The support needle 354 is formed of stainless steel, titanium, a nickel-titanium alloy (Nitinol), platinum-iridium, or a high-strength polymer, and is selected based on strength and biocompatibility. An inner diameter of the needle ranges from 0.4 to 3 mm, and is adjusted to match a diameter of the optical fiber (i.e., 0.3 to 2 mm). Accordingly, the outer diameter of the needle varies from 0.7 to 2 mm.

The first manipulation portion 351 is configured to advance/retract the optical fiber 320, and may connected to the optical fiber 320, and may also be connected to the support needle 354. The first manipulation portion 351 includes a wing unit 351a protrudes outwardly in a width direction of the handpiece cover 353, so that a user may move the optical fiber 320 connected to the first manipulation portion 351 by manipulating the wing unit 351a.

The first manipulation portion 351 is configured to only move forward and backward from the handpiece cover 353 and not move in any other direction, and a scale 353a is formed on the handpiece cover 353, thereby recognizing a movement distance of the first manipulation portion 351. The movement distance of the first manipulation portion 351 may range from 3 to 5 cm, and an interval between scale marks on the scale 353a may range from 1 to 5 mm.

The Y-shaped connector 355 may be formed with an internal space of the handpiece cover 353, but may also be formed of a tube, and may be formed from various plastic materials. The Y-shaped connector 355 is comprised of three arms. One arm is connected to a fluid supply portion 400 for inflation of the balloon 150 (see FIG. 2) or cooling of the wire basket 160 (see FIG. 5) via the fluid tube 420. Another arm is rubber-attached to allow the optical fiber 320 to move within the Y-shaped connector 355 while preventing backflow of fluid. The final arm is connected to the delivery tube 340 to allow fluid to flow through the delivery tube.

The fluid tube 420 may have a hardness ranging from Shore A to D, has an inner diameter of 1 to 3 mm and a thickness of 0.2 to 1 mm to be designed to be robust. The fluid tube 420 may be formed of Thermoplastic Polyurethane (TPU) and silicone. A control valve 421 for opening and closing the fluid tube 420 may be disposed within the fluid tube 420.

The second embodiment of the handpiece 350, illustrated in FIGS. 24 and 25, has a structure of automatically moving the optical fiber 320.

Similarly to the first embodiment, in the second embodiment of the handpiece 350, the fluid tube 420 and the optical fiber 320 are introduced, pass through the Y-shaped connector 355, and are received in an internal channel of the delivery tube 340.

The handpiece 350 includes a handpiece cover 353′ and a first manipulation portion 351. The handpiece cover 353′ forms an outer shape, and a Y-shaped connector 355 is formed inside, into which the fluid tube 420 and the optical fiber 320 are introduced and combined into the delivery tube 340. The handpiece cover 353′ includes an upper portion 353a where the tubes pass through and are combined, a grip portion 353b through which a user may grip the handpiece in a lower portion, and a switch 353c formed on the grip portion 353b to move the first manipulation portion 351.

The handpiece cover 353′ may include a handpiece cover having the same structure as the first embodiment, and a driver 357 for moving the first manipulation portion 351 of the first embodiment is disposed within the handpiece cover 353′ of the second embodiment, and thus, the switch 353c is manipulated to allow the driver 357 to operate, thereby moving the first manipulation portion 351 forward or backward. The driver 357 includes a motor, and preferably, a servo motor or stepper motor may be used to enable precise positioning, acceleration, and movement of the first manipulation portion 351. Furthermore, the motor in the driver 357 may be connected to an encoder, and the encoder provides position and speed feedback to control a movement and a final position of the first manipulation portion 351.

In the second embodiment, automatic manipulation may be performed through the driver 357 rather than manual manipulation. Furthermore, the handpiece is connected to the controller 500 (see FIG. 1) of the apparatus for laser treatment of the first embodiment, and may be manipulated by the controller 500. That is, the movement of the optical fiber 320 may be controlled by the controller based on information obtained from a sensor in the apparatus for laser treatment or a preset operating method.

Meanwhile, the third embodiment of the handpiece 350 illustrated in FIGS. 26 and 27 has a similar shape to the first embodiment and has a structure of automatically moving the optical fiber 320.

Similarly to the first embodiment, in the handpiece 350 of the third embodiment, the fluid tube 420 and the optical fiber 320 are introduced, pass through the Y-shaped connector 355, and are received in the internal channel of the delivery tube 340.

In the third embodiment, the handpiece 350 includes a handpiece cover 353″, a support needle 354, a first manipulation portion 351, and a driver 357 driving the first manipulation portion 351. The handpiece cover 353″ forms an outer shape, and a Y-shaped connector 355 is formed inside, into which the fluid tube 420 and the optical fiber 320 are introduced and combined to the delivery tube 340. The optical fiber 320 passes through the central portion of the handpiece cover 353″, and the support needle 354 supporting the passing optical fiber 320 is disposed.

The handpiece cover 353″ is provided with a switch 353c′ for manipulating the driver 357 connected to the first manipulation portion 351. In this embodiment, the switch 353c′ may be a knob operated by rotation, and depending on the forward/reverse rotation, a motor of the driver 357 may also rotate forward/reverse, thereby moving the first manipulation portion 351 forward or backward. The driver 357 may convert rotational motion into linear motion via a gear portion 358. Various mechanical systems, such as a lead screw, a rack-and-pinion system, or a belt and a pulley system, may be applied as structures for converting the motor's rotational motion into linear motion.

Although not illustrated, the rotation of the optical fiber 320 may also be implemented to operate automatically by being connected to a driver, such as a motor, and a driver driving the rotation or the driver 357 driving the linear motion may be formed into the optical fiber moving unit 310 of FIG. 1.

Furthermore, in the case of the handpiece 350 capable of automatic operation, including the driver, the handpiece may be configured to switch between a mode of operating automatically by the controller 500 (see FIG. 1) and a mode of moving the optical fiber 320 by operating switches 353c and 353c′ by the user. Accordingly, a method in which the user directly moves the optical fiber 320 after inserting the catheter 100 and irradiates light into the tubular tissue during light irradiation is possible, and a method in which light is automatically irradiated by the controller 500 is also possible.

Specifically, the present disclosure acquires shape and tissue condition information of the tubular tissue through the sensor monitoring unit 200 thereby enabling precise light irradiation by performing not only the movement of the optical fiber 320, but also the supply of fluid through the fluid management unit 400 and the irradiation of the light irradiation unit 300.

The controller 500 is connected to the robot arm 120 and includes at least one processor 501, a computer-readable storage medium 502, and a communication bus 503.

The processor 501 may cause a TMS robot control device 500 to operate according to embodiments described below. For example, the processor 501 may execute one or more programs stored in the computer-readable storage medium 502. The one or more programs may include one or more computer-executable instructions, which, when executed by the processor 501, may cause the computing device 500 to perform operations according to the embodiments.

The computer-readable storage medium 502 is configured to store computer-executable instructions, program code, program data, and/or other suitable forms of information. A program 502a stored in the computer-readable storage medium 502 includes a set of instructions executable by the processor 501.

In an embodiment, the computer-readable storage medium 502 may be a memory (a volatile memory such as a random access memory, a non-volatile memory, or a suitable combination thereof), one or more magnetic disk storage devices, optical disk storage devices, flash memory devices, any other form of storage medium capable of storing desired information and accessed by the controller 500 as another computing device, or a suitable combination thereof.

The communication bus 503 interconnects various components of the controller 500, by including the processor 501 and the computer-readable storage medium 502.

The controller 500 may also include one or more input/output interfaces 505 providing interfaces for one or more input/output devices 504, and one or more network communication interfaces 506. The input/output interfaces 505 and the network communication interfaces 506 are connected to the communication bus 503.

The input/output device 504 may be connected to other components of the controller 500 via the input/output interface 505. Exemplary input/output devices 504 may include input devices such as pointing devices (e.g., a mouse or trackpad), a keyboard, a touch input device (e.g., a touchpad or touchscreen), a voice or audio input device, various types of sensor devices, and/or a photographing device, and/or output devices such as a display device, a printer, a speaker, and/or a network card. The exemplary input/output device 504 may be incorporated into the controller 500 as a component included in the controller 500, or may be connected to the controller 500 as a separate device distinct from the controller 500.

Meanwhile, embodiments of the present disclosure may include a program for performing the methods described herein on a computer, and a computer-readable recording medium containing the program. The computer-readable recording medium may include program instructions, local data files, local data structures, and the like, either singly or in combination. The medium may be specifically designed and configured for the present disclosure, or may be commonly used in the computer software field. Examples of computer-readable recording media include magnetic media such as hard disks, floppy disks, and magnetic tapes; optical recording media such as CD-ROM and DVD; and hardware devices specifically configured to store and execute program instructions, such as ROM, RAM, and a flash memory. Examples of the program may include not only machine language code generated by a compiler, but also high-level language code that may be executed by a computer using an interpreter or the like.

Furthermore, the sensor monitoring unit 200 and the fluid management unit 400 may also include a computing device having the same configuration as the controller 500, and the sensor monitoring unit 200 and the fluid management unit 400 may be executed on the same computing device as the controller 500, or may be executed on separate computing devices.

The above-described contents present various embodiments of the apparatus for laser treatment, as well as various modified examples of balloons, wire baskets, optical fibers, and glass caps as expansion fixing portions, and it should be understood that various combinations may be achieved beyond the described embodiments, as long as they are consistent with each other.

While the present disclosure has been described above with reference to the embodiments described, it should be understood that the present disclosure is not limited to the embodiments described above, and that modifications and variations may be made by those skilled in the art without altering the technical concept of the present disclosure as claimed in the claims.

What is claimed is:

1. An apparatus for laser treatment, comprising:
a light irradiation unit including an optical fiber connected to a light source;
a catheter having an expansion fixing portion into which the optical fiber is inserted, and which fixes a position thereof in a tubular tissue within a body;
a sensor monitoring unit including a plurality of sensors provided in the catheter; and
a controller receiving measurement information from the sensor monitoring unit and controlling the light source of the light irradiation unit, and including a processor and a storage medium connected to the processor and storing a control program,
wherein the plurality of sensors include a shape information sensor inserted together with the optical fiber and configured to obtain internal shape information of the tubular tissue in a position of an optical fiber end to which light is irradiated, and
in a cross-section, perpendicular to a longitudinal direction of the catheter, the shape information sensor is disposed in a central portion, together with the optical fiber.

2. The apparatus for laser treatment according to claim 1, wherein the catheter includes a transparent tube surrounding the optical fiber and a delivery tube surrounding the transparent tube, and
the sensor monitoring unit includes a shape information sensor wire connected to the shape information sensor, and
the shape information sensor wire is disposed inside the delivery tube.

3. The apparatus for laser treatment according to claim 2, wherein the expansion fixing portion includes a balloon into which the optical fiber is inserted and which is inflated by a fluid, and
the shape information sensor includes a piezoelectric sensor.

4. The apparatus for laser treatment according to claim 3, wherein the piezoelectric sensor is disposed in plural in a longitudinal direction of the catheter within the balloon.

5. The apparatus for laser treatment according to claim 3, wherein the piezoelectric sensor is disposed further from an end of the catheter than the optical fiber end.

6. The apparatus for laser treatment according to claim 5, further comprising:
a fluid management unit including a pump for supplying fluid to inflate the balloon and removing the supplied fluid, and
the control program performs an operation of controlling the light source and an amount of fluid supplied from the fluid management unit based on a measurement value of the piezoelectric sensor.

7. The apparatus for laser treatment according to claim 6, wherein the fluid management unit includes an air trap remover so as to remove an air trap inside the balloon and a cooler generating a cooling fluid for lowering a temperature of the fluid.

8. The apparatus for laser treatment according to claim 3, wherein the delivery tube has a plurality of channels, a transparent tube is disposed in one of the plurality of channels, and the shape information sensor wire is disposed in another channel.

9. The apparatus for laser treatment according to claim 8, wherein the plurality of sensors include a temperature sensor, and
the temperature sensor is attached to the balloon.

10. The apparatus for laser treatment according to claim 2, further comprising:
a cooling liquid supply portion supplying a cooling liquid to the optical fiber end through a cooling channel disposed within the delivery tube,
wherein the cooling channel includes an outlet disposed closer to an end of the catheter than the optical fiber end,
the expansion fixing portion includes a wire basket configured to be contractible and expandable, and
the optical fiber end is disposed within the wire basket.

11. The apparatus for laser treatment according to claim 10, wherein the wire basket includes a plurality of wires, and
at least a portion of the plurality of sensors are attached to the plurality of wires.

12. The apparatus for laser treatment according to claim 11, wherein the sensor attached to the wire includes at least one of a pH measurement sensor or a mucosal impedance measurement sensor.

13. The apparatus for laser treatment according to claim 10, wherein the expansion fixing portion further includes a balloon inflated by fluid in a different position from the wire basket in a longitudinal direction of the catheter.

14. The apparatus for laser treatment according to claim 13, wherein the balloon is disposed further from the end of the catheter than the wire basket.

15. The apparatus for laser treatment according to claim 3, wherein a light irradiation portion includes a glass cap covering the optical fiber end, and
the optical fiber is one of a diffusion optical fiber, a radial optical fiber and a side-firing fiber.

16. The apparatus for laser treatment according to claim 15, wherein the glass cap has a circular or polygonal inner surface in a longitudinal cross-section of the optical fiber.

17. The apparatus for laser treatment according to claim 3, further comprising:
a handpiece manipulating a movement of the catheter,
wherein the piezoelectric sensor is cylindrical,
the optical fiber is a side-firing fiber, and
the handpiece includes a rotational manipulation unit connected to the optical fiber and the piezoelectric sensor and rotating the optical fiber and the piezoelectric sensor.

18. An apparatus for laser treatment, comprising:
a light irradiation unit including an optical fiber connected to a light source;
a catheter including a flexible tip disposed in a tip portion thereof, a balloon disposed behind the flexible tip, into which the optical fiber is inserted, and which fixes a position in a tubular tissue within a body, and an endoscope unit disposed in back of the balloon;
a piezoelectric sensor attached to the optical fiber within the balloon and configured to obtain diameter information of the tubular tissue behind an optical fiber end to which light is irradiated; and
a fluid management unit including a pump for supplying fluid to inflate the balloon and removing the supplied fluid,
wherein the piezoelectric sensor is disposed in plural in a longitudinal direction of the catheter,
the piezoelectric sensor is disposed in a central portion, together with the optical fiber, in a cross-section, perpendicular to the longitudinal direction of the catheter,
the catheter includes a transparent tube surrounding the optical fiber and a delivery tube surrounding the transparent tube, and
the delivery tube includes a channel through which fluid passes, a channel in which the transparent tube is disposed, and a channel in which a wire connected to the piezoelectric sensor is disposed.

19. The apparatus for laser treatment according to claim 18, further comprising:
a controller connected to the light source connected to the light irradiation unit, the piezoelectric sensor and the fluid management unit, and including a processor and a storage medium connected to the processor and storing a control program, and
the control program performs an operation of controlling the light source and the fluid management unit based on a measurement value of the piezoelectric sensor.

20. The apparatus for laser treatment according to claim 19, further comprising:
a moving portion including a motor for moving the catheter,
wherein the controller is connected to the moving portion.
